(19) United States
(12) Patent Application Publication
(10) Pub. No.: US 2025/0177056 A1
(43) Pub. Date: Jun. 5, 2025

(54) THREE-DIMENSIONAL RECONSTRUCTION OF AN INSTRUMENT AND PROCEDURE SITE

(71) Applicant: AURIS HEALTH, INC., Santa Clara, CA (US)

(72) Inventors: Menglong YE, Mountain View, CA (US); Hedyeh RAFII-TARI, Mountain View, CA (US)

(21) Appl. No.: 18/841,205

(22) PCT Filed: Feb. 23, 2023
(86) PCT No.: PCT/IB2023/051682

§ 371 Date: Aug. 23, 2024

Related U.S. Application Data
(60) Provisional application No. 63/313,350, filed on Feb. 24, 2022.

(51) Int. Cl. A61B 34/20; A61B 90/00

(52) U.S. Cl. CPC A61B 34/20; A61B 90/37; A61B 2034/2048; A61B 2090/3764

(57) ABSTRACT

The present disclosure relates to systems, devices, and methods to reconstruct a three-dimensional model of an instrument and a procedure site using trained neural networks.

### RELATED APPLICATION(S)

This application claims priority to U.S. Provisional Application No. 63/313,350, filed Feb. 24, 2022, entitled THREE-DIMENSIONAL RECONSTRUCTION OF AN INSTRUMENT AND PROCEDURE SITE and PCT/IB2023/051682, filed Feb. 23, 2023, entitled THREE-DIMENSIONAL RECONSTRUCTION OF AN INSTRUMENT AND PROCEDURE SITE, the disclosures of each of which are hereby incorporated by references in their entireties.

### BACKGROUND

Various medical procedures involve the use of one or more devices configured to penetrate the human anatomy to reach a treatment site. Certain operational processes can involve localizing a medical instrument within the patient and visualizing an area of interest within the patient. To do so, many medical instruments may include sensors to track the location of the instrument and may include vision capabilities, such as embedded cameras or the compatible use with vision probes.

### BRIEF DESCRIPTION OF THE DRAWINGS

Various embodiments are depicted in the accompanying drawings for illustrative purposes and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIG. 1 is a block diagram that illustrates an example medical system for performing various medical procedures in accordance with aspects of the present disclosure.

FIG. 2 is a diagram illustrating components and subsystems of the control system shown in FIG. 1, according to an example embodiment.

FIG. 3 is a flowchart illustrating a method to reconstruct a three-dimensional model of an instrument and a procedure site from two-dimensional images acquired during or as part of a medical procedure, according to an example embodiment.

FIG. 4 is a system diagram illustrating a neural network generation system 400, according to an example embodiment.

FIG. 5 is a flowchart illustrating a method to generate a trained neural network usable to reconstruct a three-dimensional model of an instrument and a procedure site, according to an example embodiment.

FIG. 6 is block diagram illustrating an example data architecture for fluoroscope image processing, according to an example embodiment.

FIG. 7 is a diagram illustrating a series of instrument segmentations, according to an example embodiment, that may be produced by the neural network.

FIG. 8 is a diagram illustrating segmentations of an instrument that includes subparts, scope tip and articulatable section, according to an example embodiment.

FIG. 9 illustrates an example of a segmentation of the procedure site, according to example embodiments.

FIG. 10 is a diagram illustrating a segmentation that is based on a region of interest, such as a distance centered around an area of a segmented instrument, according to an example embodiment.

FIG. 11 is a diagram illustrating a calibration object, according to an example embodiment.

FIG. 12 is a diagram illustrating example calibration images, according to an example embodiment.

FIG. 13 is a diagram illustrating a reconstructed 3D model rendering, according to an example embodiment

### DETAILED DESCRIPTION

The headings provided herein are for convenience only and do not necessarily affect the scope or meaning of disclosure. Although certain exemplary embodiments are disclosed below, the subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Overview

The present disclosure relates to systems, devices, and methods to generate reconstructed three-dimensional models of a procedure site and an instrument. Herein, the term reconstruct can be understood to mean construct, and vice versa. Many medical procedures rely on accurate representations of a patient's anatomy and navigation in controlling instruments within that anatomy. For example, in bronchoscopy, accurate and safe biopsy may depend on accurate alignment between a steerable bronchoscope and a biopsy site, such as a nodule or lesion. Robotic bronchoscopy can include a navigation system to facilitate navigation of the bronchoscope to the biopsy site and provide information helpful in aligning the tip of the bronchoscope with the biopsy site. The navigation system may include a three-dimensional model of the anatomy. In the case of bronchoscopy, the three-dimensional model may include data regarding the structure of the luminal network formed by the airways of the lung. This three-dimensional model can be generated from a preoperative computerized tomography (CT) scan of the patient. During the procedure, the coordinate system of the three-dimensional model is registered with the coordinate system of a location sensor (or location sensors) incorporated in the bronchoscope so that the navigation system can provide an estimate of the bronchoscope's location within the luminal network of the lungs. Examples of location sensors include robotized sensors, inertial measurement units (IMUs), fiber-optic shape sensors, electromagnetic (EM) sensors, and camera sensors.

Location sensors have their limitation when being used to provide the navigation functionality. For example, the accuracy of robotized sensors may suffer from their miniaturized sizes, the accuracy of IMUs may suffer from accumulated errors, the accuracy of fiber-optic shape sensors may be affected by environmental temperature, the accuracy of EM sensors may suffer from ferro-magnetic objects and the localization accuracy of camera sensors may suffer from low quality images. In this regard, interventional imaging modalities such as a fluoroscopic/x-ray scanning device may be used to provide additional contextual information of a robotic bronchoscope inside patient body.

However, fluoroscopic images are two-dimensional by nature and understanding the volumetric morphology of an anatomy from a single or even a series of fluoroscopic images can be challenging for a physician. Embodiments described herein may reconstruct three-dimensional models of an instrument and a procedure site from a limited set of two-dimensional images. Rendering the reconstructed three-dimensional models may provide a user interface to the operator that provides three-dimensional context to the operator, which allows better insight when aligning the bronchoscope tip to the biopsy site. Additionally or alternatively, the reconstructed model may be registered to the preoperative model and this registration may facilitate improvements to the navigation system.

### Three-Dimensional Pose Estimation System

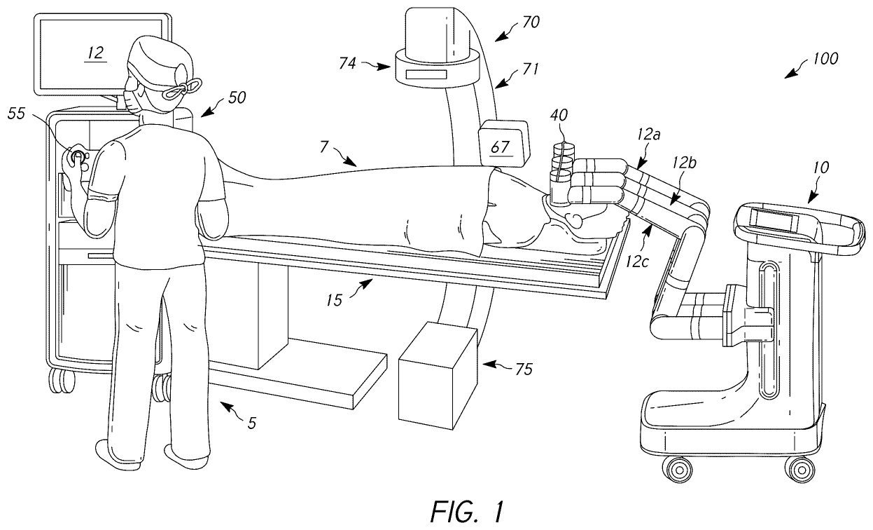

FIG. 1 illustrates an example medical system 100 for performing various medical procedures in accordance with aspects of the present disclosure. The medical system 100 may be used for, for example, endoscopic procedures. Robotic medical solutions can provide relatively higher precision, superior control, and/or superior hand-eye coordination with respect to certain instruments compared to strictly manual procedures. Although the system 100 of FIG. 1 is presented in the context of a bronchoscopy procedure, it should be understood that the principles disclosed herein may be implemented in any type of endoscopic procedure.

The medical system 100 includes a robotic system 10 (e.g., mobile robotic cart) configured to engage with and/or control a medical instrument (e.g., bronchoscope) including a proximal handle 31 and a shaft 40 coupled to the handle 31 at a proximal portion thereof to perform a procedure on a patient 7. It should be understood that the instrument 40 may be any type of shaft-based medical instrument, including an endoscope (such as a ureteroscope or bronchoscope), catheter (such as a steerable or non-steerable catheter), needle, nephroscope, laparoscope, or other type of medical instrument. The instrument 40 may access the internal patient anatomy through direct access (e.g., through a natural orifice) and/or through percutaneous access via skin/tissue puncture.

The medical system 100 includes a control system 50 configured to interface with the robotic system 10, provide information regarding the procedure, and/or perform a variety of other operations. For example, the control system 50 can include one or more display(s) 56 configured to present certain information to assist the physician 5 and/or other technician(s) or individual(s). The medical system 100 can include a table 15 configured to hold the patient 7. The system 100 may further include an electromagnetic (EM) field generator, such as a robot-mounted EM field generator 80 or and EM field generator 85 mounted to the table 15 or other structure.

Although the various robotic arms 12 are shown in various positions and coupled to various tools/devices, it should be understood that such configurations are shown for convenience and illustration purposes, and such robotic arms may have different configurations over time and/or at different points during a medical procedure. Furthermore, the robotic arms 12 may be coupled to different devices/instruments than shown in FIG. 1, and in some cases or periods of time, one or more of the arms may not be utilized or coupled to a medical instrument. Instrument coupling to the robotic system 10 may be via robotic end effectors 6 associated with distal ends of the respective arms 12. The term “end effector” is used herein according to its broad and ordinary meaning and may refer to any type of robotic manipulator device, component, and/or assembly. The terms “robotic manipulator” and “robotic manipulator assembly” are used according to their broad and ordinary meanings and may refer to a robotic end effector and/or sterile adapter or other adapter component coupled to the end effector, either collectively or individually. For example, “robotic manipulator” or “robotic manipulator assembly” may refer to an instrument device manipulator (IDM) including one or more drive outputs, whether embodied in a robotic end effector, adapter, and/or other component(s).

In some embodiments, the physician 5 can interact with the control system 50 and/or the robotic system 10 to cause/control the robotic system 10 to advance and navigate the medical instrument shaft 40 (e.g., a scope) through the patient anatomy to the target site and/or perform certain operations using the relevant instrumentation. The control system 50 can provide information via the display(s) 56 that is associated with the medical instrument 40, such as real-time endoscopic images captured therewith, and/or other instruments of the system 100, to assist the physician 5 in navigating/controlling such instrumentation. The control system 50 may provide imaging/positional information to the physician 5 that is based on certain positioning modalities, such as fluoroscopy, ultrasound, optical/camera imaging, EM field positioning, or other modality, as described in detail herein.

The various scope/shaft-type instruments disclosed herein, such as the shaft 40 of the system 100, can be configured to navigate within the human anatomy, such as within a natural orifice or lumen of the human anatomy. The terms “scope” and “endoscope” are used herein according to their broad and ordinary meanings and may refer to any type of elongate (e.g., shaft-type) medical instrument having image generating, viewing, and/or capturing functionality and being configured to be introduced into any type of organ, cavity, lumen, chamber, or space of a body. A scope can include, for example, a ureteroscope (e.g., for accessing the urinary tract), a laparoscope, a nephoscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise an at least partially rigid and/or flexible tube, and may be dimensioned to be passed within an outer sheath, catheter, introducer, or other lumen-type device, or may be used without such devices. Endoscopes and other instruments described herein can have associated with distal ends or other portions thereof certain markers/sensors configured to be visible/detectable in a field/space associated with one or more positioning (e.g., imaging) systems/modalities.

The system 100 is illustrated as including an imaging device (e.g., a fluoroscopy system) 70, which includes an X-ray generator 75 and an image detector 74 (referred to as an “image intensifier” in some contexts; either component 74, 75 may be referred to as a “source” herein), which may both be mounted on a moveable C-arm 71. The control system 50 or other system/device may be used to store and/or manipulate images generated using the imaging device 70. In some embodiments, the bed 15 is radiolucent, such that radiation from the generator 75 may pass through the bed 15 and the target area of the patient's anatomy, wherein the patient 7 is positioned between the ends of the C-arm 71. The structure/arm 71 of the fluoroscopy system 70 may be rotatable or fixed. The imaging device 70 may be implemented to allow live images to be viewed to facilitate image-guided surgery. The structure/arm 71 can be selectively moveable to permit various images of the patient 7 and/or surgical field to be taken by the fluoroscopy panel source 74.

In the example bronchoscopy configuration shown in FIG. 1, the field generator 67 is mounted to the bed. In other example embodiments, the field generator 67 may be mounted to a robotic arm. As the electric field generated by the electric field generator 67 can be distorted by the presence of metal or other conductive components therein, it may be desirable to position the electric field generator 67 in a manner such that other components of the system do not interfere substantially with the electric field. For example, it may be desirable to position the electric field generator 67 at least 8″ or more away from the support arm 71 associated with the fluoroscopy system.

The system 100 (as with other systems disclosed herein) can include an optical imaging source (not shown), such as a camera device (e.g., stereoscopic camera assembly, a depth sensing camera assembly (e.g., RGB/RGBD)). The optical imaging source may be configured/used to view a field in the surgical environment to identify certain marker(s) disposed in the visual field. For example, in some embodiments, the imaging source may emit infrared (IR) or other-frequency electromagnetic radiation and/or detect reflection of such radiation to identify markers that include surfaces that reflect such radiation. Such optical deflection can indicate position and/or orientation of the marker(s) associated with the particular optical modality. The system 100 can have certain markers/fiducials which may be detectable/positionable in one or more reference/coordinate frames/spaces associated with respective positioning modalities.

As shown in FIG. 1, the image detector 74 may include one or more external tracking sensors 78. The external tracking sensors 78 include a location sensor as described above, optical tracking, depth sensing cameras, or some combination thereof usable to determine the pose of the imaging device 70.

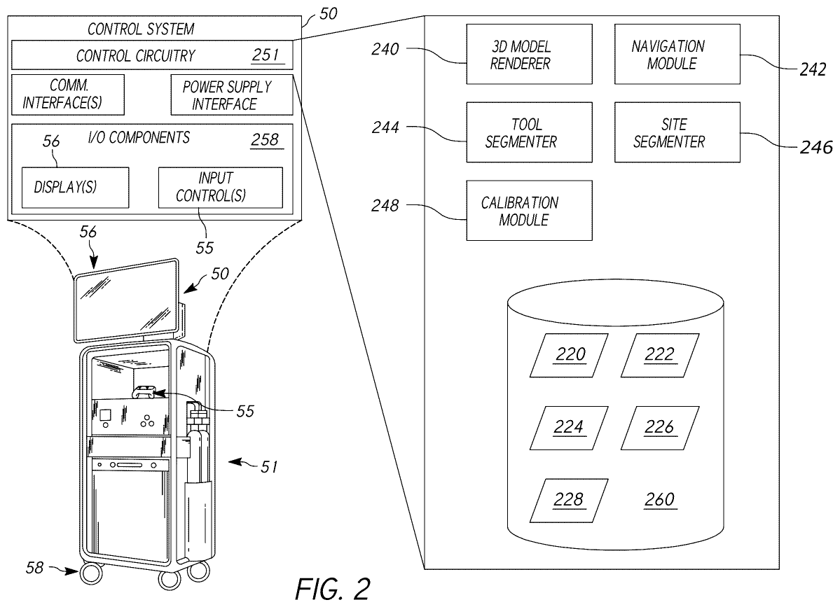

FIG. 2 is a diagram illustrating components and subsystems of the control system 50 shown in FIG. 1, according to an example embodiment. As discussed above, the control system 50 can be configured to provide various functionality to assist in performing a medical procedure. The control system 50 can communicate with the robotic system 10 via a wireless or wired connection (e.g., to control the robotic system 10). In some embodiments, the control system 50 can communicate with the robotic system 10 to receive position data therefrom relating to the position of the distal end of the scope 40 or other instrumentation. Such positioning data may be derived using one or more location sensors (e.g., electromagnetic sensors, shape sensing fibers, accelerometers, gyroscopes, satellite-based positioning sensors (e.g., a global positioning system (GPS)), radio-frequency transceivers, and so on) associated with the respective instrumentation and/or based at least in part on robotic system data (e.g., arm position/pose data, known parameters or dimensions of the various system components, etc.) and vision-based algorithms. In some embodiments, the control system 50 can communicate with the EM field generator to control generation of an EM field in an area around the patient 7 and/or around the tracked instrumentation.

As referenced above, the system 100 can include certain control circuitry configured to perform certain of the functionality described herein, including the control circuitry 251 of the control system 50. That is, the control circuitry of the systems 100 may be part of the robotic system 10, the control system 50, or some combination thereof. Therefore, any reference herein to control circuitry may refer to circuitry embodied in a robotic system, a control system, or any other component of a medical system, such as the medical systems 100 shown in FIG. 1, respectively. The term “control circuitry” is used herein according to its broad and ordinary meaning, and may refer to any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 251 may be entirely locally maintained/disposed or may be remotely located at least in part (e.g., communicatively coupled indirectly via a local area network and/or a wide area network). Any of the control circuitry 251 may be configured to perform any aspect(s) of the various processes disclosed herein.

With further reference to FIG. 2, the control system 50 can include various I/O components 258 configured to assist the physician 5 or others in performing a medical procedure. For example, the input/output (I/O) components 258 can be configured to allow for user input to control/navigate the scope 40 and/or basketing system within the patient 7. In some embodiments, for example, the physician 5 can provide input to the control system 50 and/or robotic system 10, wherein in response to such input, control signals can be sent to the robotic system 10 to manipulate the scope 40 and/or other robotically-controlled instrumentation.

The control system 50 and/or robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore. Such user controls are communicatively and/or physically coupled to respective control circuitry. The control system can include a structural tower 51, as well as one or more wheels 58 that support the tower 51. The control system 50 can further include certain communication interface(s) 254 and/or power supply interface(s) 259.

The control circuitry 251 may include a data store 260 that stores various types of data, such as intraoperative images 220, trained networks 222, sensor data 224, calibration data 226, and preoperative 3D model 228. The intraoperative images 220 may be data representing images acquired during a procedure, as for example, via a fluoroscopic scanner, cone-beam or C-arm scanner. The intraoperative images may be a two-dimensional image that shows the 2D positioning of an instrument and a procedure site. As discussed in greater detail later in this disclosure, intraoperative images 220 may be used as inputs to generate 3D representations of these positionings to provide more insight on proper 3D alignment between the instruments and the procedure site.

The trained networks 222 may include data and logic configured to identify one or both of an instrument or procedure site depicted in an intraoperative image. In some embodiments, the trained networks 222 may include one or more trained networks that only segment an instrument and then another set of one or more trained networks that only segment a procedure site. In other embodiments, the trained networks 222 may include networks configured to identify both an instrument (or instruments) and a procedure site. As is discussed in greater detail below, the trained networks may be generated by a network training system that is communicatively or electrically coupled with the control system 50.

The sensor data 224 may include raw data gathered from and/or processed by input devices (e.g., control system 50, optical sensor, EM sensor, IDM) for generating estimated state information for the instrument as well as output navigation data. By way of example and not limitation, the sensor data 224 may include image data, location sensor data, robot data. Image data may include one or more image frames captured by the imaging device at the instrument tip, as well as information such as frame rates or timestamps that allow a determination of the time elapsed between pairs of frames. Robot data includes data related to physical movement of the medical instrument or part of the medical instrument (e.g., the instrument tip or sheath) within the tubular network. Example robot data includes command data instructing the instrument tip to reach a specific anatomical site and/or change its orientation (e.g., with a specific pitch, roll, yaw, insertion, and retraction for one or both of a leader and a sheath) within the tubular network, insertion data representing insertion movement of the part of the medical instrument (e.g., the instrument tip or sheath), IDM data, and mechanical data representing mechanical movement of an elongate member of the medical instrument, for example motion of one or more pull wires, tendons or shafts of the endoscope that drive the actual movement of the medial instrument within the tubular network. Location sensor data may include data collected by location sensors of the instruments (e.g., EM sensors, shape sensing fiber, and the like).

The calibration data 226 may include data representing intrinsic parameters of the imaging device, such as principal points, focal lengths, distortion factors, and the like. The calibration data 226 may be obtained by an imaging device calibration procedure.

The preoperative three-dimensional model data 228 may be a computer-generated 3D model representing an anatomical space, according to one embodiment. The preoperative three-dimensional model data 228 may be generated using centerlines that were obtained by processing CT scans that were generated preoperatively. In some embodiments, computer software may be able to map a navigation path within the tubular network to access a procedure site within the model represented by the preoperative three-dimensional model data.

Various module of the control circuitry 251 may process the data stored in the data store 221. For example, the control circuitry may include a 3D model renderer 240, a navigation module 242, a tool segmenter 244, a site segmenter 246, and a calibration module 248. The tool segmenter 244 may process, using one or more trained networks (e.g., trained networks 222), intraoperative image data to generate segmented data corresponding to the instrument depicted in the intraoperative image data. Similar to the tool segmenter 244, the site segmenter 246 may process intraoperative image data to generate segmented data corresponding to the procedure site depicted in the intraoperative image data, also using one or more trained networks (e.g., trained networks 222). It is to be appreciated that, although the tool segmenter 244 and the site segmenter 246 are shown in FIG. 2 as being separate modules, other embodiments may have a single module that performs the functions of both the tool segmenter 244 and the site segmenter 246. Generally, the segmented data produced by the segmenters 244, 246 may feed into the input of the 3D model renderer, which is discussed below. However, some embodiments, the segmented data may be provided to an operator of the system 100 to be rendered in a way that is helpful for the operator. As discussed below, a segmented stream may be provided to the operator as they make real-time adjustments to the placement of the instrument.

The 3D model renderer 240 may be control circuitry configured to process the segmented intraoperative image data and reconstruct a 3D rendering of the instrument relative to the procedure site.

The navigation module 242 processes various data (e.g., sensor data 224) and provides the localization data of the instrument tip as a function of time, where the localization data indicates the estimated position and orientation information of the instrument tip within an anatomy. In some embodiments, the navigation module 242 registers a coordinate frame corresponding to the location sensor of the instrument to a coordinate frame of the preoperative 3D model 228.

### Three-Dimensional Model Reconstruction Methods and Operations

Details of the operations of exemplary model reconstruction systems are now discussed. The methods and operation disclosed herein are described relative to the model reconstruction systems 100 shown in FIG. 1 and the modules and other components shown in FIG. 2. However, it is to be appreciated that the methods and operations may be performed by any of the components, alone or in combination, discussed herein.

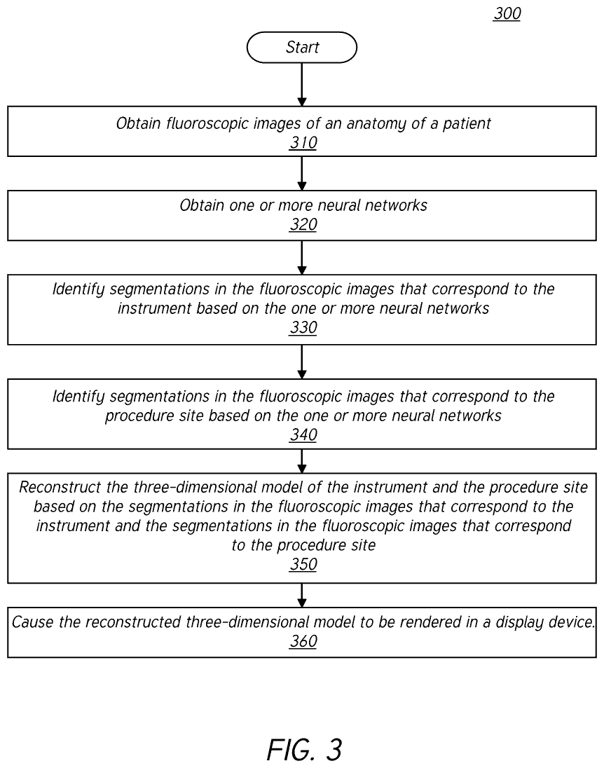

A model reconstruction system may generate a representation of a three-dimensional anatomy based on a comparatively limited number of two-dimensional images acquired during or as part of a medical procedure. FIG. 3 is a flow-chart illustrating a method 300 to reconstruct a 3D model of an instrument and a procedure site from two-dimensional images acquired during or as part medical procedure, according to an example embodiment. As FIG. 3 shows, the method 300 may begin at block 310, where the system 100 obtains two-dimensional images (e.g., fluoroscopic images) of an anatomy of a patient obtained by the imaging device 70. In some embodiments, a domain transfer occurs wherein the imaging device 70 is configured to replicate desired characteristics. As an example, some embodiments may configure the imaging device 70 to adjust principal points, focal lengths, distortion factors, or any other parameter of the imaging device.

At block 320, the system obtains one or more neural networks previously trained by images generated based on one or more computerized tomography scans. As used herein, a system or apparatus may “obtain” data in any number of mechanisms, such as push or pull models or access through local storage. For example, a neural network service (described in below) may send a neural network or neural networks to the control system 50 based on determinable events (e.g., determining a neural network has been updated or a new one is available) or based on a schedule that updates the control system's neural networks periodically. In other examples, the control system 50 may send a request to a neural network service for a neural network and, in response to the request, the neural network service may respond by sending the requested neural network to the control system 50. Still further, some embodiments of the control system may generate their own local copies of the neural networks and may obtain the neural network at block 320 via retrieving them from local storage devices, such as data store 260.

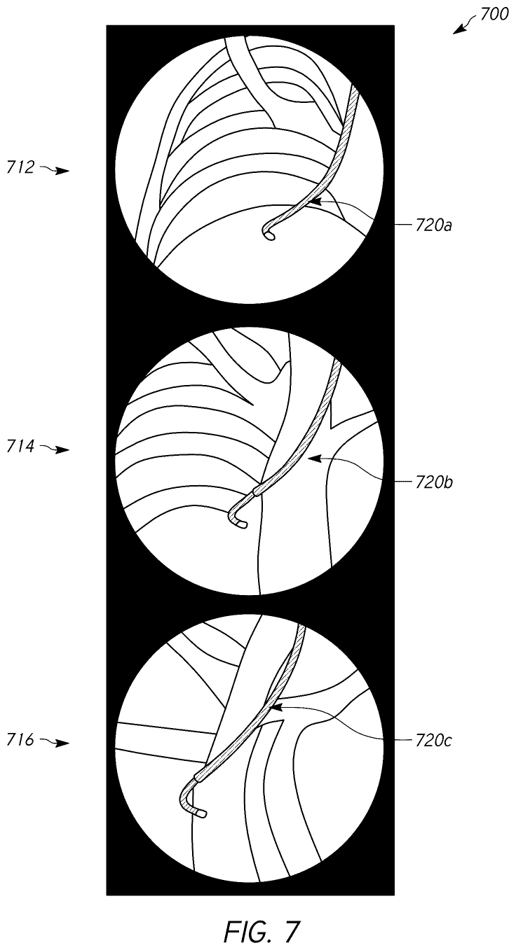

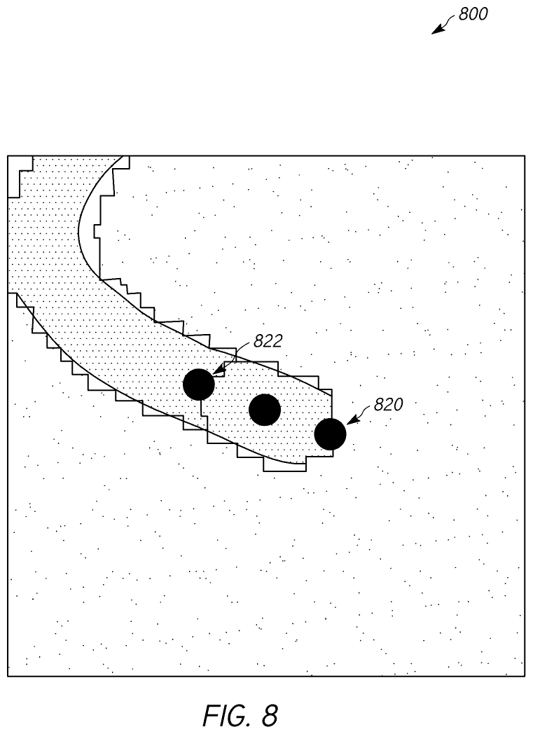

At block 330, the system uses the one or more neural networks to identify segmentations in the two-dimensional images (e.g., fluoroscopic images) obtained at block 310 that correspond to the instrument. Examples of segmentations of two-dimensional images that correspond to the instrument are shown with reference to FIGS. 7 and 8. FIG. 7 is a diagram illustrating a series of instrument segmentations 700, according to an example embodiment, that may be produced by the neural network. Each of the fluoroscopic images 712, 714, 716 are obtained from the imaging device 70 pointed at the same anatomical location of the patient but at different angles. As an example and not a limitation, image 712 may be taken at a 15-degree left anterior oblique view. Image 714 may be taken at a 0-degree anteroposterior view. Image 716 may be taken at a 15-degree right anterior oblique. Each of the images 712, 714, 716 includes a respective segmentation 720a, 720b, 720 of the instrument. In some embodiments, the segmentation includes sub-segments representing detectable parts of the instrument. For example, FIG. 8 is a diagram illustrating segmentations of an instrument that includes subparts, scope tip 820 and articulatable section 822, according to an example embodiment. Further, in some embodiments, directional relationship can be determined based on the relationship between the subparts.

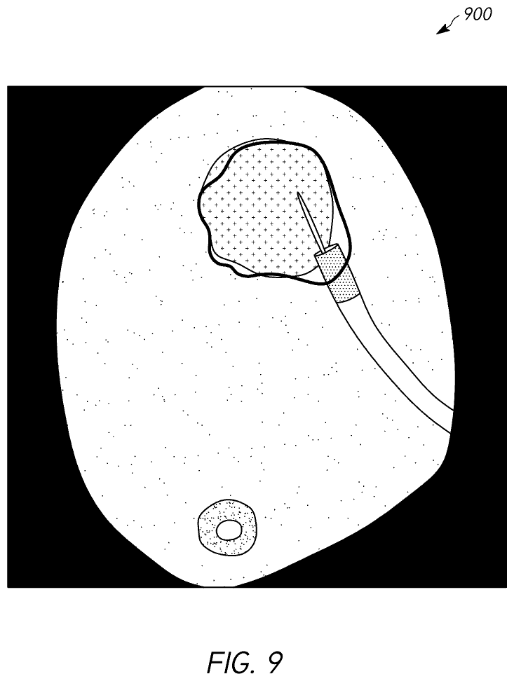

With reference back to FIG. 3, at block 340, the system 100 may use the one or more neural networks to identify segmentations in the fluoroscopic images that correspond to the procedure site (e.g., a tumor or legion). FIG. 9 illustrates an example of a segmentation of the procedure site, according to example embodiments. For example, FIG. 9 is a segmentation of a fluoroscope image wherein a tumor nodule is segmented from the rest of the image.

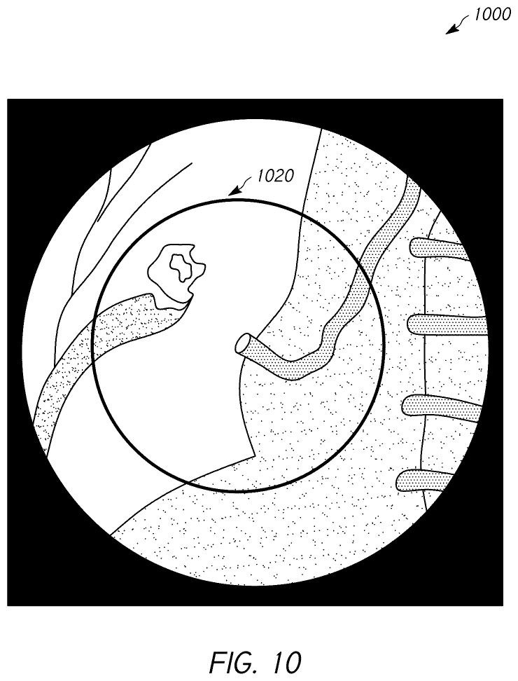

With reference back to FIG. 3, it is to be appreciated that the order and timing of executing blocks 330 and 340 is flexible. For example, blocks 330 and 340 can be executed in parallel or in sequence. In some embodiments, block 340 can be executed dependent of the outcome of block 330. For example, the instrument segmentation at block 330 may be used to generate a region-of-interest (ROI) that can then be used for facilitating finer procedure site segmentation of block 340. That is, the system will segment in a determinable area around the instrument or an identifiable part of the instrument, such as the instrument tip. FIG. 10 is a diagram illustrating a segmentation that is based on a region of interest 1020, such as a distance centered around an area of a segmented instrument, according to an example embodiment. Example distances include 3 centimeters but can be any suitable distance based on the context of the procedure.

With reference back to FIG. 3, at block 350, the system may reconstruct a three-dimensional model of the instrument and the procedure site using the segmentations in the two-dimensional images that correspond to the instrument and the segmentations in the two-dimensional images that correspond to the procedure site. Reconstruction may take two or more two-dimensional images. Although it is contemplated that two two-dimensional images will provide sufficient accuracy, many embodiments may choose to increase the number of two-dimensional images, say three or more. For example, one embodiment may reconstruct using three two-dimensional images, one from a 15-degree left anterior oblique view, a second from a 0-degree anteroposterior view, and a third from a 15-degree right anterior oblique view.

The reconstruction can be performed using triangulation of the different two-dimensional images and intraoperative imaging device poses and imaging device intrinsic parameters. The imaging device intrinsic parameters (the principal point, focal lengths, and distortion factors) can be obtained via a camera calibration procedure. The intraoperative imaging device poses can be retrieved in multiple ways: if a motorized scanner is used, the pose, at which a fluoroscopic image is taken, can be obtained from the provided output of the imaging device; if the pose information is not provided by the system, the external tracking sensors 78 of FIG. 1 (optical tracking; inertial measurement unit; RGB/RGBD cameras) can then be placed on the scanner to obtain the pose information. In some embodiments, the system may instruct the operator to obtain images at predefined angles and the system can assume that the operator followed the directions.

### Neural Network Training

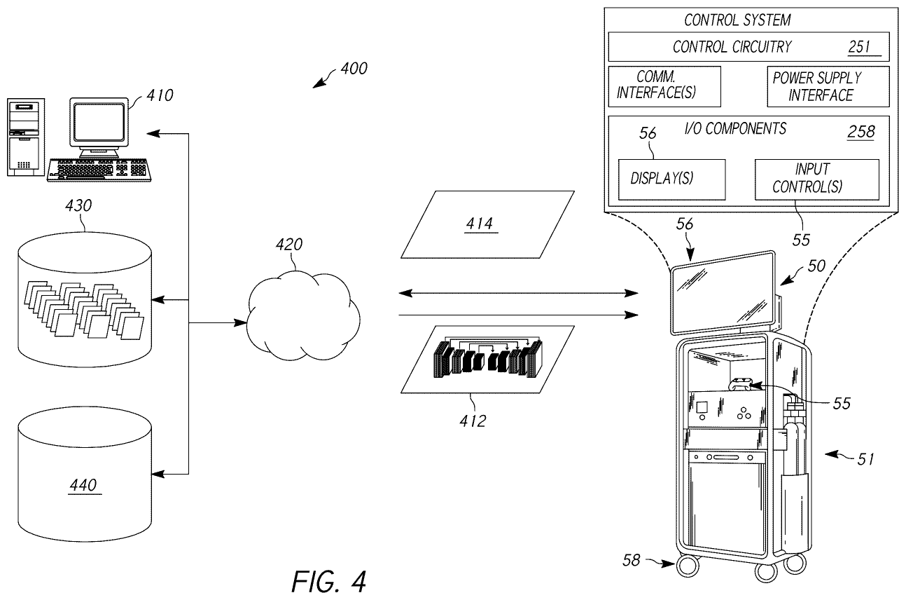

As discussed above, with reference to block 320 of FIG. 3, the system 100 may obtain neural networks trained on by images from one or more CT scans. Also discussed above, the neural network may be accessed through a neural network service. The concepts involving the generation of neural network and a neural network service are now described in greater detail. FIG. 4 is a system diagram illustrating a neural network generation system 400, according to an example embodiment. The neural network generation system 400 may include networked communication between the control system 50 of FIG. 1 and a neural network training system 410 via network 420. The network 420 may include any suitable communication network that allows two or more computer systems to communicate with each other, which can include a wireless and/or wired network. Example networks include one or more personal area networks (PANs), local area networks (LANs), wide area networks (WANs), Internet area networks (IANs), cellular networks, the Internet, etc.

The neural network training system 410 may be a computer system configured to generate neural networks 412 usable to generate reconstructed three-dimensional models of an instrument and a procedure site from intraoperative two-dimensional images (e.g., fluoroscopy images). Although the neural network training system 410 shown in FIG. 4 communicates to the control system 50 via the network 420, it is to be appreciated that the neural network training system 410 may also be onsite relative to the control system and may operate in accordance with the preoperative software of associated with the control system. The preoperative software of the control system 50 may be software usable to generate a preoperative three-dimensional model of a patient's anatomy, plan a path to a procedure site, and the like.

The neural network training system 410 may be coupled to a two-dimensional image database 430. The two-dimensional image database 430 may include annotated images of an anatomy, such as a lung or a kidney. The CT scans may include known shape and location of tumors. In an example, the shape may be known based on manual annotation included by an experienced human observer or based on automatic annotations from computer-based vision algorithms. In some cases, alternative or additionally to the pool of patients, the two-dimensional image database 430 may include a CT scan of a patient in which a procedure is being planned.

The neural network training system 410 may be coupled also to an instrument model database 440. The instrument model database 440 includes data characterizing known geometrical properties of the instruments, which may be stored, for example, in a computer-aided design file.

As shown, the neural network training system 410 and the control system 50 may exchange domain data. Domain data 414 may be data that characterizes an operation based on a calibration of the imaging device or the patient. The neural network training system 410 may use the domain data 414 to specialize the data being used to train the neural networks that are sent to the control system 50.

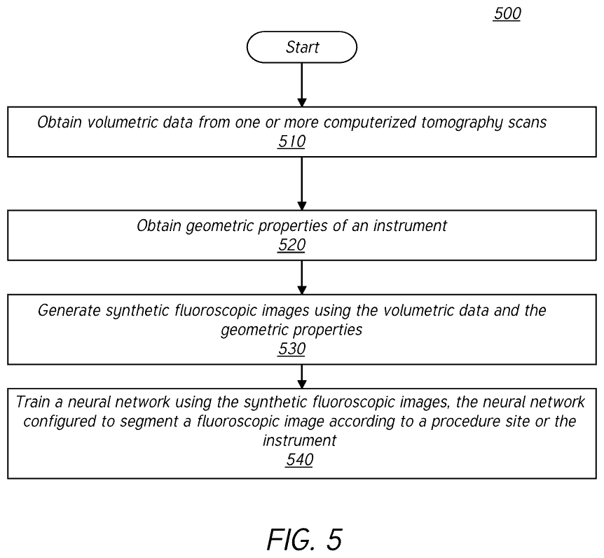

FIG. 5 is a flowchart illustrating a method 500 to generate a trained neural network usable to reconstruct a three-dimensional model of an instrument and a procedure site, according to an example embodiment. The method 500 may begin at block 510 where the neural network training system 410 obtains volumetric data from one or more CT scans. In some embodiments, the neural network training system 410 may select the one or more CT scans based on the domain data 414, where the domain data 414 characterizes the patient, such as based on age, race, gender, physical condition, and the like. In other embodiments, the one or more CT scans includes a CT scan acquired from the patient as part of a preoperative planning procedure.

At block 520, the neural network training system 410 obtains geometric properties of an instrument. As discussed above, the geometric properties of the instrument may include geometric data derived from a CAD file.

At block 530, the neural network training system 410 generates synthetic fluoroscopic images using the volumetric data and the geometric properties. In one embodiment, as part of block 530, the neural network training system 410 utilizes the patient-specific CT scan data to generate digitally reconstructed radiology (DRR) images. The inputs for DRR data generation include the known 3D shape and location of the procedure site (e.g., tumor/nodule), and the known instrument geometrical properties (available in the CAD file). Based on sampling the viewing angles and distances in the 3D space of the CT scan coordinate frame, DRR data generation can be done using back projections/ray-tracing techniques or deep learning-based techniques. The generated DRR images will share similar image properties as real fluoroscopic images. To further generated DRR images that share similar image properties as the real fluoroscopic images, the settings and example images generated by the imaging device may be provided as part of the domain transfer. The neural network training system 410 may use these settings and example images to generate the DRR images.

At block 540, the neural network training system 410 trains a neural network using the synthetic fluoroscopic images. The neural network, once trained, is configured to segment a fluoroscopic image according to a procedure site or the instrument. In the example discussed above where, at block 530, the neural network training system 410 generates DRR images from the CT scans, these DRR images can be used as synthetic fluoroscopic images to train neural networks for instrument segmentation and procedure site segmentation. The architecture of the segmentation networks can be based on convolutional neural networks (e.g., such as UNet and ResNet), transformers or graph neural networks.

The neural network training system 410 then makes the trained neural network, or networks, available to the control system 50. The neural network training system 410 may make the neural network available by providing access through the network 420 or by providing transfer via a computer readable medium, where the neural network training system 410 and the control system 50 are collocated.

### Example Overall Framework

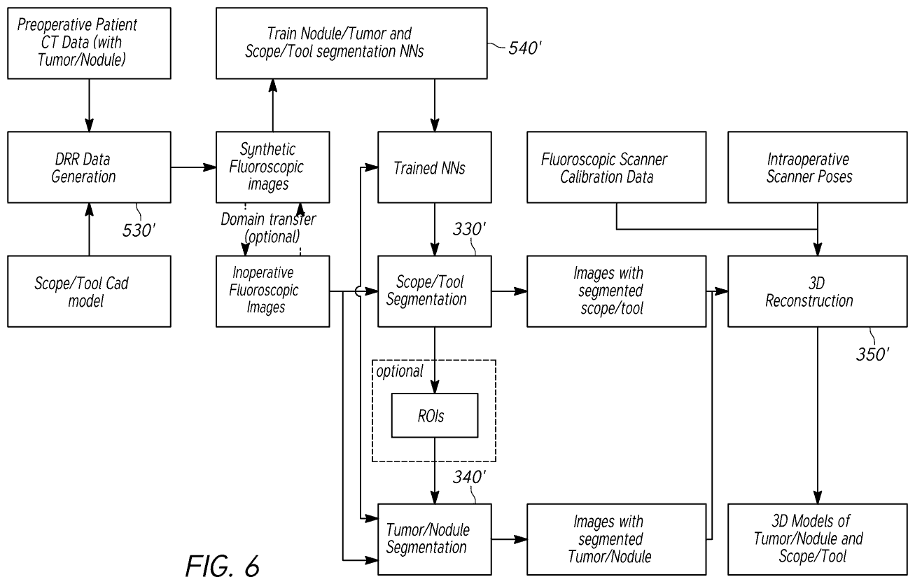

FIG. 6 is block diagram illustrating an example data architecture 600 for fluoroscope image processing, according to an example embodiment. The functional blocks of the example data architecture 600 are labelled according to the corresponding block from the flowcharts discussed above. As FIG. 6 shows, there is domain data that is transferred or exchanged as part of the synthetic fluoroscopic images and the intraoperative fluoroscopic images. This is so that the synthetic fluoroscopic images can be generated by a neural network training system to accurately reflect the intraoperative fluoroscopic images that will be generated by a medical system performing the medical procedure. For example, the domain data may include the principal points focal lengths, distortion factors, and of the imaging device 70 of FIG. 1. The generated DRRs may than reflect these parameters to more closely align the images that the imaging device 70 is likely to produce.

Likewise, where the imaging device has capabilities for configuring itself, the domain data may include data usable by the medical system to configure the imaging device. Examples of domain data that may be used by the control system include imaging angles, image contrast, depth, and the like.

It is also to be appreciated that the example data architecture 600 shown in FIG. 6 also illustrates that the segmenters 330′ and 340′ may be used in a correlated fashion. That is, the instrument segmenter 330′ may first segment an instrument from an inoperative image and then the system may identify a region of interest based on the instrument segmentation. Based on the region of interest, the procedure site segmenter 340′ may then produce a refined segmentation of the procedure site based on the area within the region of interest.

### Navigation Integration

As discussed above, with reference to FIG. 2, the control system 50 may include a navigation system that localizes an instrument to a patient via a 3D preoperative model. After the control system generates a reconstructed 3D model of the instrument and procedure site, the control system may register the reconstructed 3D model of the instrument and procedure site to the CT/patient frame of reference. Once registered, the segmented 3D scope or 3D nodule information can be used as another input into the navigation module to improve the accuracy of the navigation output, which is also in the CT/patient frame of reference.

The information can be used in any number of ways. For example, the control system 50 may use the 3D reconstructed scope pose information as another input into the Navigation “Fusion” framework to combine with the other outputs from the EM-based, Vision-based, and robot-based algorithms, to improve the accuracy of the output of the Fusion framework.

As another example, the control system 50 may use the 3D reconstructed nodule to correct for intraoperative nodule location estimation and updating nodule location. This can help compensate for errors that are caused by CT-body divergence and anatomical deformation caused intraoperatively.

As another example, the control system 50 may use the 3D reconstructed scope and nodule location to calculate the relative distance between the scope tip and nodule and use this information to correct the distance-to-target measurements displayed to the user.

As another example, the control system 50 may use the 3D reconstructed shape of the scope to input shape information into the Navigation framework. The shape information can be registered against the skeleton/path to provide more stable navigation information vs. just the scope tip position.

As another example, the control system 50 may use the shape information from the scope to detect and model intraoperative anatomy deformation. In some embodiments, the control system can adaptively update the 3D map/lung model based on the shape information of the scope.

### Segmentation Streams

In some situations, once the operator has the instrument near the procedure site, the operator may elect to take fluoroscopic images to confirm the pose of the instrument relative to the procedure site. Although much of this disclosure discusses embodiments that produce reconstructed 3D models of the instrument and the procedure site, these and other embodiments may render intermediary results to aid the operator in making fine-tuned adjustments of the instrument. For example, in some embodiments, the control system may render the intraoperative fluoroscopic images to the operator with data identifying the different segmentations in the intraoperative fluoroscopic images. Augmenting the intraoperative fluoroscopic images with segmentation data may provide highlighted imagery of the instrument and the procedure site which can be helpful for the operator in visualizing the pose of the instrument even though the intraoperative images are only in 2D. This is especially useful where the operator puts the imaging device in a mode that provides fluoroscopic images in rapid succession or video like appearance, referred herein as an “intraoperative image stream”. With an intraoperative image stream, the processing time to reconstruct the 3D models of the instrument and procedure site may be longer than the rate in which the intraoperative images are captured. Thus, where a stream of 2D intraoperative images is provided, the control system may render a segmented view of the stream of intraoperative images, where the segmented views include visual indicators of the segmented instrument and segmented procedure site. As used herein, a view of the stream of intraoperative images augmented with segmentation data may be referred to herein as a “segmentation stream.”

This 2D visualization of the procedure site can be shown on a fluoroscopic image taken from different angles based on pose information of the imaging device at the time in which the 2D intraoperative image is taken. The control system may acquire the pose information (or some aspect of pose, such orientation or location) manually (e.g., a date entered by an operator of the system via a user interface provided by the control system), via a communication interface between the control system and the imaging device, or external tracking sensors. The control system may then back project the 3D model of the procedure site onto the 2D intraoperative image using camera parameters and the pose information.

### Imaging Device Calibration

Fluoroscopic/X-ray images acquired by a C-Arm scanner can be used to reconstruct the 3D anatomical scene. The reconstruction module of the control system 50 (see FIG. 2) may use intrinsic and extrinsic parameters of the C-Arm scanner to achieve comparatively accurate 3D reconstruction. As previously discussed, a C-Arm scanner includes a source generator (that emits x-ray source) and a detector that identifies the x-ray to create an image. The calibration module may treat this source-detector imaging mechanism as a pinhole camera model, and thus the intrinsic parameters would be principal points, focal lengths and distortion factors. If the C-arm scanners have flat-panel detectors, there would be no distortion on the acquired fluoroscopic/x-ray images, and therefore the distortion factors can be neglected in the calibration process.

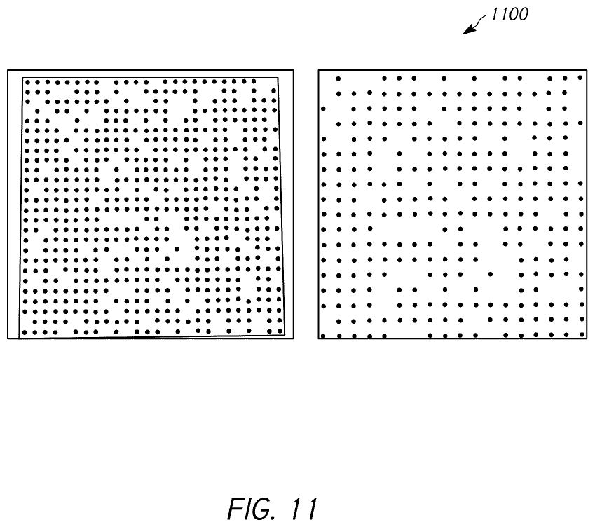

To calculate the intrinsic parameters of a fluoroscopic/x-ray scanner, a calibration object for the calibration module can be designed to facilitate for this. FIG. 11 is a diagram illustrating a calibration object 1100, according to an example embodiment The calibration object 1100 can be a planar object with small-sized balls attached on it. The material used for making the calibration object 1100 may have low opacity under fluoroscopic imaging such that the metallic balls can be clearly seen by the imaging device. The rectangular geometrical pattern of the ball placement on the calibration object 1100 can be randomly generated before tool machining, and this known geometrical pattern will be then used in the calibration process.

i. Intrinsic Calibration

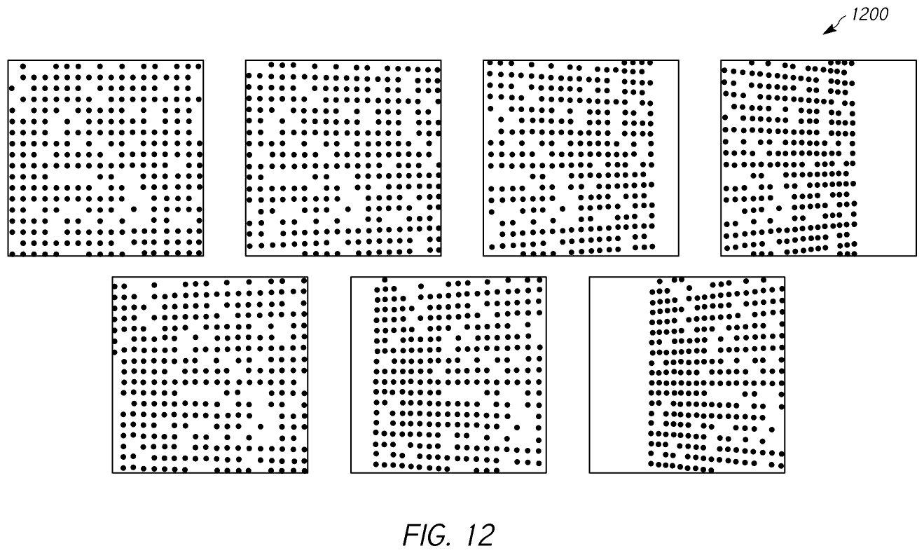

The following discusses intrinsic calibration. For data collection, an operator running the calibration process may place the calibration object on the patient bed and adjust the height of the fluoroscopic scanner such that the calibration object is roughly at the center between the source generator and detector. Then the scanner is rotated with various pre-defined angles and at each angle take a fluoroscopic image on the calibration object. Example images 1200 of these data are provided in FIG. 12.

For data processing, for any image collected, the calibration module detects the metallic ball locations on the image of the calibration object, and this can be performed by, for example, blob detection algorithms. After obtaining the ball locations on an image of the calibration object, the calibration module matches these detected balls to the correspondences in the known geometrical pattern of the calibration object. This matching process can have following steps:
1: Calculate pairwise distances between ball locations, loop over each ball location and perform:
1a: at each ball location, gather the top X (e.g., 8) nearest-neighbor locations.
2: Find the dominant orientations on the image
2a: At each ball location, create the vectors from it to its 8 nearest-neighbor locations, respectively.
2b: Calculate the orientations of the vectors, and this then outputs a histogram of orientations (Here, the orientations range from 0 to 180 degrees, that means 225 will be assigned as 45 degrees).
2c: Identify two orientations from the orientation histogram, which are corresponding to horizontal and vertical directions on the known geometrical pattern.
3: Create a graph by checking the ball locations and their nearest neighbors. The cost of a connection between two vertices in the graph can be defined as a combination of their location distance and orientation.
4: Find the largest connected component of the graph and match this largest connected component inside the known geometrical pattern. After graph matching, the location correspondences between the detected balls and the pattern are determined.

The calibration module performs the above correspondence searching between all collected images and the known geometrical pattern. These obtained correspondences are then used to obtain the intrinsic parameters that includes principal points, focal lengths and distortion factors (optional) via a camera calibration algorithm.

i. Extrinsic Calibration

For extrinsic calibration, most fluoroscopic/x-ray scanners do not provide the 6 Degree-of-Freedom (DoF) pose information (orientation and translation) during imaging. To acquire this information, an external tracking sensor can be used. This external tracking sensor can be either an IMU device or RGB-D camera (or a combination of the two). This external tracking sensor generates the 6 DoF pose data on its own coordinate frame. In order to transform the pose data into the scanner imaging coordinate frame, a hand-to-eye calibration can be performed.

To gather the data for this calibration process, the external tracking sensor is attached to the scanner, preferable close to the detector. Then the system performs fluoroscopic/x-ray imaging on the calibration object at various angles/translations where the object is at least partially visible in the fluoroscopic images. At each pose when the image is taken, the calibration module records the pose output from the external tracking sensor. With these paired image-and-pose dataset, the calibration module can then perform a hand-eye calibration algorithm to calculate the senor-to-scanner transformation. This transformation can then be used intraoperative to transform the readings from the sensor to the scanner imaging coordinate frame, thus providing the pose information needed for 3D scene reconstruction.

### Example Three-Dimensional Reconstruction

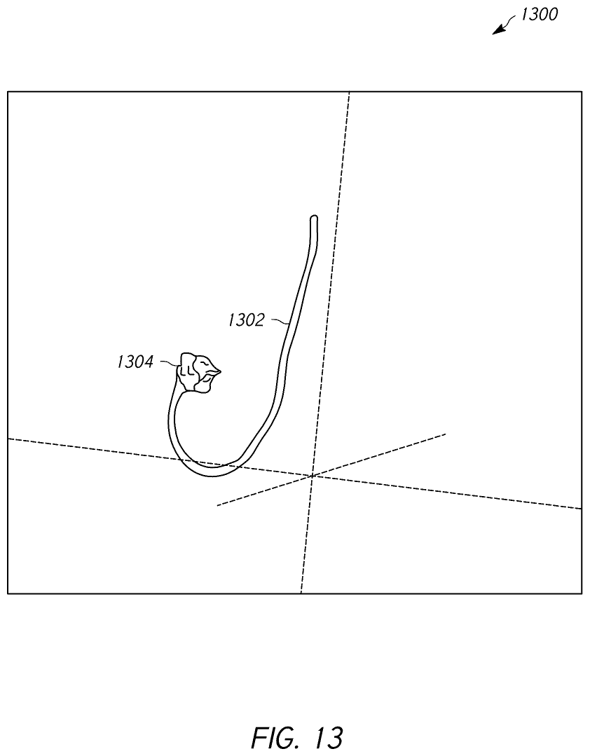

As discussed above with reference to block 360 of FIG. 3, the control system may cause the reconstructed 3D model to be rendered in a display device. FIG. 13 is a diagram illustrating a reconstructed 3D model rendering 1300, according to an example embodiment. The reconstructed 3D model rendering 1300 may include data that, when rendered by a display device, shows the shape and pose of an instrument 1302 relative to a procedure site 1304. The methods and systems for generating the reconstructed 3D models are described in greater detail above.

Although not shown, in some embodiments, if a reconstructed 3D model is registered to a virtual model of an anatomy (e.g., such as a lung, kidney, gastrointestinal system), features of the virtual model of the anatomy may be depicted in conjunction (e.g., overlayed) with the reconstructed 3D model.

Although not shown, in some embodiments, the reconstructed 3D model rendering 1300 may include user interface features that facilitate alignment of the instrument and the procedure site. For example, in one embodiment, the reconstructed 3D model rendering 1300 may include a graphical element such as a line (patterned or not) extending axially from the tip of the instrument 1302. In these embodiments, the operator may cause the robotic system to adjust the pose of the instrument until the line extending from the instrument 1302 shown in the rendering 1300 intersects with the procedure site 1304. In some embodiments, the rendering 1300 may be updated by the system when certain events are detected, such as alignment between the instrument 1302 and the procedure site 1304. One such update may be to change the color of the procedure site or a graphical element extending from the instrument 1302 depending on the alignment between the instrument and the procedure site. For example, the procedure site 1304 may be updated to be rendered in one color when there is no alignment and another color when there is an alignment. In some embodiments, the color may represent a strength of the alignment, so that the operator can distinguish between an alignment that may result in sampling an edge of a nodule or sampling a center of a nodule.

### Implementing Systems and Terminology

Implementations disclosed herein provide systems, methods and apparatus to reconstruct a three-dimensional model of an anatomy using two-dimensional images. Various implementations described herein provide for improved visualization of an anatomy during a medical procedure using a medical robot.

The system 100 can include a variety of other components. For example, the system 100 can include one or more control electronics/circuitry, power sources, pneumatics, optical sources, actuators (e.g., motors to move the robotic arms), memory, and/or communication interfaces (e.g., to communicate with another device). In some embodiments, the memory can store computer-executable instructions that, when executed by the control circuitry, cause the control circuitry to perform any of the operations discussed herein. For example, the memory can store computer-executable instructions that, when executed by the control circuitry, cause the control circuitry to receive input and/or a control signal regarding manipulation of the robotic arms and, in response, control the robotic arms to be positioned in a particular arrangement.

The various components of the system 100 can be electrically and/or communicatively coupled using certain connectivity circuitry/devices/features, which can or may not be part of the control circuitry. For example, the connectivity feature(s) can include one or more printed circuit boards configured to facilitate mounting and/or interconnectivity of at least some of the various components/circuitry of the system 100. In some embodiments, two or more of the control circuitry, the data storage/memory, the communication interface, the power supply unit(s), and/or the input/output (I/O) component(s), can be electrically and/or communicatively coupled to each other.

The term “memory” is used herein according to its broad and ordinary meaning and can refer to any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

Computer-readable media that can be implemented in accordance with embodiments of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Described herein are systems, devices, and methods to generate reconstructed three-dimensional models of a procedure site and an instrument. Some implementations of the present disclosure relate to a method comprising obtaining volumetric data from one or more computerized tomography (CT) scans labeled according to parts of an anatomy, obtaining geometric properties of an instrument, generating synthetic fluoroscopic images based on the volumetric data and the geometric properties, and training one or more neural networks using the synthetic fluoroscopic images. The neural network can be configured to segment a fluoroscopic image according to a procedure site or the instrument.

In some embodiments, the one or more neural networks can include a first neural network configured to segment the fluoroscopic image according to the procedure site and a second neural network configured to segment the fluoroscopic image according to the instrument. In some embodiments, the one or more neural networks includes a single neural network configured to segment the fluoroscopic image according to both the procedure site and the instrument.

In some embodiments, the method can further include obtaining domain data corresponding to a procedure to be performed on a patient by a medical system. In some embodiments, the domain data can include at least one of a principal point, a focal length, or a distortion factor. In some embodiments, generating the synthetic fluoroscopic images can be based further on the domain data.

In some embodiments, generating the synthetic fluoroscopic images can be based on superimposing a representation of the instrument on the synthetic fluoroscopic images based on the geometric properties and a preoperative path. In some embodiments, the CT scans can lack a representation of the instrument. In some embodiments, the procedure site can be a lung nodule. In some embodiments, generating the synthetic fluoroscopic images can include generating a first synthetic fluoroscopic image focused at a portion of an anatomy at a first angle and a second synthetic fluoroscopic image focused at a portion of an anatomy at a second angle different from the first angle. In some embodiments, generating the synthetic fluoroscopic images can further include generating a third synthetic fluoroscopic image focused at the portion of an anatomy at a third angle different from the first angle and the second angle.

Some implementations of the present disclosure relate to a system to train one or more neural networks usable to segment intraoperative fluoroscopic images, the system comprising control circuitry and a computer-readable medium. The computer-readable medium can have instructions that, when executed, cause the control circuitry to obtain at least one of volumetric data from one or more computerized tomography (CT) scans labeled according to parts of an anatomy and geometric properties of an instrument, generate synthetic fluoroscopic images based on at least one of the volumetric data and the geometric properties, and train the one or more neural networks using the synthetic fluoroscopic images. The neural network can be configured to segment the intraoperative fluoroscopic image according to a procedure site or the instrument.

Some implementations of the present disclosure relate to a method to reconstruct a three-dimensional model of an instrument and a procedure site within an anatomy. The method can comprise obtaining fluoroscopic images of an anatomy of a patient, obtaining one or more neural networks, identifying segmentations in the fluoroscopic images that correspond to the instrument based on the one or more neural networks, identifying segmentations in the fluoroscopic images that correspond to the procedure site based on the one or more neural networks, reconstructing the three-dimensional model of the instrument and the procedure site based on the segmentations in the fluoroscopic images that correspond to the instrument and the segmentations in the fluoroscopic images that correspond to the procedure site, and causing the reconstructed three-dimensional model to be rendered in a display device.

In some embodiments, the method can further include determining a region-of-interest based on the segmentations in the fluoroscopic images that correspond to the instrument. The identifying of the segmentations in the fluoroscopic images that correspond to the procedure site can be based on the region-of-interest. In some embodiments, the identifying of the segmentations in the fluoroscopic images that correspond to the instrument can be performed in parallel with the identifying of the segmentations in the fluoroscopic images that correspond to the procedure site. In some embodiments, the reconstructing of the three-dimensional model of the instrument and the procedure site can be further based on calibration data derived from the imaging device that generated the fluoroscopic images of the anatomy of the patient.

In some embodiments, the method can further include causing the segmentations in the fluoroscopic images that correspond to the instrument and the segmentations in the fluoroscopic images that correspond to the procedure site to be rendered in the display device.

In some embodiments, the segmentations in the fluoroscopic images that correspond to the procedure site and the segmentations in the fluoroscopic images that correspond to the instrument can be rendered in the display device prior to the reconstructed three-dimensional model being rendered in the display device. In some embodiments, the segmentations in the fluoroscopic images that correspond to the instrument can include a first sub-segmentation and a second sub-segmentation, wherein the first sub-segmentation and the second sub-segmentation correspond to different components of the instrument.

In some embodiments, the procedure site can correspond to a biopsy site. In some embodiments, the fluoroscopic images of the anatomy of the patient can include a first fluoroscopic image focused on the anatomy at a first angle and a second fluoroscopic image focused on the anatomy at a second angle different from the first angle.

In some embodiments, reconstructing the three-dimensional model of the instrument and the procedure site can include triangulating segments identified in the first fluoroscopic image and segments identified in the second fluoroscopic image.

In some embodiments, the method can further include obtaining at least one of the first angle or the second angle via a communication interface of an imaging device that generated the first and second fluoroscopic images. In some embodiments, the method can further include obtaining at least one of the first angle or the second angle via an operator user interface. In some embodiments, the method can further include obtaining at least one of the first angle or the second angle via an external tracking sensor.

Some implementations of the present disclosure relate to a system to reconstruct a three-dimensional model of an instrument and a procedure site within an anatomy. The system can comprise control circuitry and a computer-readable medium. The computer-readable medium can have instructions that, when executed, cause the control circuitry to: obtain fluoroscopic images of an anatomy of a patient, obtain one or more neural networks, identify segmentations in the fluoroscopic images that correspond to the instrument based on the one or more neural networks, identify segmentations in the fluoroscopic images that correspond to the procedure site based on the one or more neural networks, reconstruct the three-dimensional model of the instrument and the procedure site based on the segmentations in the fluoroscopic images, and cause the reconstructed three-dimensional model to be rendered in a display device.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. may be either X, Y, or Z. Thus, such conjunctive language is not generally intended to imply that certain embodiments require at least one of X, at least one of Y, and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the disclosure should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

1. A method, the method comprising:
obtaining volumetric data from one or more computerized tomography (CT) scans labeled according to parts of an anatomy;
obtaining geometric properties of an instrument;
generating synthetic fluoroscopic images based on the volumetric data and the geometric properties; and
training one or more neural networks using the synthetic fluoroscopic images, the neural network configured to segment a fluoroscopic image according to a procedure site or the instrument.

2. The method of claim 1, wherein the one or more neural networks includes a first neural network configured to segment the fluoroscopic image according to the procedure site and a second neural network configured to segment the fluoroscopic image according to the instrument.

3. (canceled)

4. The method of claim 1, further comprising obtaining domain data corresponding to a procedure to be performed on a patient by a medical system.

5. The method of claim 4, wherein the domain data includes at least one of: a principal point, a focal length, or a distortion factor.

6. The method of claim 4, wherein generating the synthetic fluoroscopic images is based further on the domain data.

7. The method of claim 1, wherein generating the synthetic fluoroscopic images is based on superimposing a representation of the instrument on the synthetic fluoroscopic images based on the geometric properties and a preoperative path.

8. The method of claim 1, wherein the CT scans lack a representation of the instrument.

9. (canceled)

10. The method of claim 1, wherein generating the synthetic fluoroscopic images includes generating a first synthetic fluoroscopic image focused at a portion of an anatomy at a first angle and a second synthetic fluoroscopic image focused at a portion of an anatomy at a second angle different from the first angle.

11. The method of claim 10, wherein generating the synthetic fluoroscopic images further includes generating a third synthetic fluoroscopic image focused at the portion of an anatomy at a third angle different from the first angle and the second angle.

12. A system to train one or more neural networks usable to segment intraoperative fluoroscopic images, the system comprising:
control circuitry;
a computer-readable medium, the computer-readable medium having instructions that, when executed, cause the control circuitry to:
obtain at least one of volumetric data from one or more computerized tomography (CT) scans labeled according to parts of an anatomy and geometric properties of an instrument,
generate synthetic fluoroscopic images based on at least one of the volumetric data and the geometric properties, and
train the one or more neural networks using the synthetic fluoroscopic images, the neural network configured to segment the intraoperative fluoroscopic image according to a procedure site or the instrument.

13. A method to reconstruct a three-dimensional model of an instrument and a procedure site within an anatomy, the method comprising:
obtaining fluoroscopic images of an anatomy of a patient;
obtaining one or more neural networks;
identifying segmentations in the fluoroscopic images that correspond to the instrument based on the one or more neural networks;
identifying segmentations in the fluoroscopic images that correspond to the procedure site based on the one or more neural networks;
reconstructing the three-dimensional model of the instrument and the procedure site based on the segmentations in the fluoroscopic images that correspond to the instrument and the segmentations in the fluoroscopic images that correspond to the procedure site; and
causing the reconstructed three-dimensional model to be rendered in a display device.

14. The method of claim 13, further comprising: determining a region-of-interest based on the segmentations in the fluoroscopic images that correspond to the instrument, wherein the identifying of the segmentations in the fluoroscopic images that correspond to the procedure site is based on the region-of-interest.

15. The method of claim 13, wherein the identifying of the segmentations in the fluoroscopic images that correspond to the instrument is performed in parallel with the identifying of the segmentations in the fluoroscopic images that correspond to the procedure site.

16. The method of claim 13, wherein the reconstructing of the three-dimensional model of the instrument and the procedure site is further based on calibration data derived from the imaging device that generated the fluoroscopic images of the anatomy of the patient.

17. The method of claim 13, further comprising causing the segmentations in the fluoroscopic images that correspond to the instrument and the segmentations in the fluoroscopic images that correspond to the procedure site to be rendered in the display device.

18. The method of claim 17, wherein the segmentations in the fluoroscopic images that correspond to the procedure site and the segmentations in the fluoroscopic images that correspond to the instrument are rendered in the display device prior to the reconstructed three-dimensional model being rendered in the display device.

19. The method of claim 13, wherein the segmentations in the fluoroscopic images that correspond to the instrument include a first sub-segmentation and a second sub-segmentation, wherein the first sub-segmentation and the second sub-segmentation correspond to different components of the instrument.

20. (canceled)

21. The method of claim 13, wherein the fluoroscopic images of the anatomy of the patient includes a first fluoroscopic image focused on the anatomy at a first angle and a second fluoroscopic image focused on the anatomy at a second angle different from the first angle.

22. The method of claim 21, wherein reconstructing the three-dimensional model of the instrument and the procedure site comprises triangulating segments identified in the first fluoroscopic image and segments identified in the second fluoroscopic image.

23. (canceled)

24. (canceled)

25. The method of claim 21, further comprising obtaining at least one of the first angle or the second angle via an external tracking sensor.

26. (canceled)
